(19) United States
(12) Patent Application Publication
(10) Pub. No.: US 2021/0219979 A1
(43) Pub. Date: Jul. 22, 2021

(54) ENDOSCOPE ACCESSORY DEVICES

(71) Applicant: SHAARE ZEDEK SCIENTIFIC LTD., Jerusalem (IL)

(72) Inventors: Tomer ADAR, Ramat Hasharon (IL); Eran GOLDIN, Tel Aviv (IL)

(21) Appl. No.: 15/734,717

(22) PCT Filed: Jun. 3, 2019
(86) PCT No.: PCT/IL2019/050629

§ 371 Date: Dec. 3, 2020

Related U.S. Application Data
(60) Provisional application No. 62/680,643, filed on Jun. 5, 2018.

(51) Int. Cl. A61B 17/12; A61B 17/34; A61B 90/00

(52) U.S. Cl. CPC A61B 17/12013; A61B 2017/00818; A61B 90/39; A61B 17/3478

(57) ABSTRACT

The disclosure provides endoscopic accessory devices and methods of using same, the accessory devices having an elongated shaft with a tissue engagement member at its distal end, the tissue engagement member configured to encircle gastrointestinal tissue when applied thereagainst.

### TECHNICAL FIELD

The present disclosure generally relates to the field of endoscopy, more particularly to endoscopic cuffs configured to provide an external working channel for use with an endoscope and accessory devices for use therewith.

### BACKGROUND

Endoscopes are presently used for screening, diagnostic, and therapeutic purposes.

There are many different uses for endoscopes, and while the general design of different models is similar, some variations exist to optimize the performance of the endoscope for its intended purpose.

The endoscope may include and/or allow passage of one or more diagnostic or treatment devices.

Conventional endoscopes, such as the endoscope illustratively depicted in FIG. 1, have an endoscope probe 100 connected at its proximal end to a handle (not shown). Probe 100 is adapted to be inserted into a patient's gastrointestinal lumen (but may also apply to non-digestive lumens and other body cavities) to perform selected therapeutic, diagnostic, and/or screening procedures. Probe 100 typically contains an imaging system 110 having optical fibers, or the like, extending along the length of the probe, a light guide 120, an air/water spray nozzle 130 and a working channel 140.

One example of an endoscopic procedure is Endoscopic Variceal Ligation (EVL). Esophageal varices may develop as a result of increased pressure in the portal venous system, often as a complication of liver disease and cirrhosis. During EVL (also called rubber band ligation) an enlarged vein or varix in the esophagus is tied off or ligated by rubber bands delivered by a ligator positioned on an endoscope.

However, due to the position of the ligator on the endoscope's distal end, the field of view of the endoscope's imaging system may be obstructed and thus complicate the procedure. Furthermore, commonly used ligation devices have a finite amount of rubber bands, and in cases where additional bands are required, the entire endoscope needs to be extracted for mounting a new device, and then reintroduced. Finally, since commonly used ligation devices use an endoscope-mounted cup for suctioning prior to band application to the base of the varix, the circumference of suctioned tissue is limited by the cup size, which is limited to fit the endoscope circumference.

Another example of an endoscopic procedure is mucosal marking, often performed by tattooing. This may be required in various clinical indications, including to identify a location of a lesion for further endoscopic surveillance, indication of a lesion's location to guide surgical resections, and others.

Endoscopic tattooing is typically considered to be of low risk; however, an optimal technique is needed to ensure its success (visible marking at the correct location) and prevent possible complications caused by transmural/too deep injections (with possible marking in adjacent bowel loops) or invisible lesions (as a result of superficial injections or injection into the mesenteric or retroperitoneal side of the intestine).

The number of complications following endoscopic tattooing is mostly caused by transmural injection, which may result in adverse effects including focal peritonitis, infected hematoma and/or abscess formation, inflammatory pseudotumor, idiopathic inflammatory bowel disease, post-operative adhesions, and tumor inoculation, as have been published.

In addition, inaccurate landmarking may require need for prolonged operative time and reoperation due to lack of oncologic resection.

There is thus a need for endoscopic accessory devices, which facilitate the performing of endoscopic procedures in an accurate and visible manner, thus reducing associated complications.

### SUMMARY

The present disclosure is directed to endoscopic accessory devices.

Inter alia, the disclosure is directed to an endoscopic accessory device configured for Endoscopic Variceal Ligation (EVL). The device includes an elongated shaft having a ring/loop shaped tissue engagement member at its distal end. The tissue engagement member is configured to encircle an esophageal varix and to allow ligating bands to be released around the encircled varix so as to encompass and compress its base, thus facilitating its ligation. Advantageously, the accessory device may be passed through a working channel of the endoscope or through a working channel of an endoscopic cuff encaging the endoscopic probe, as further elaborated hereinbelow. Resultingly, the need for an endoscopic cup and ligator (as in current standard practice), mounted on the distal end of the endoscopic probe, is obviated. This is of uttermost importance since the ligator cup tends to interfere with endoscopic field of vision (thus complicating the procedure), restricts the tissue that can be suctioned, and restricts the band size and number that can be used in each endoscopic insertion.

The disclosure also provides an endoscopic accessory device for endoscopic tattooing of gastrointestinal tissue, such as, but not limited to, colon tissue. The device includes an elongated shaft having a ring/loop-shaped tissue engagement member at its distal end and a needle configured to be extended horizontally into the space encircled by the tissue engagement member so as to pierce into the tissue encaged by the tissue engagement member. This allows accurate, plain-oriented delivery of the ink and thus tattooing of the target tissue (e.g. a colorectal lesion).

Currently, endoscopic tattooing requires that the injection needle be inserted about 3 cm from the lesion at an angle to the colon wall ensuring that the tip of the needle is beneath the mucosa. The needle must then be pulled back to the submucosa level. Elevating the tissue is helpful to verify submucosa depth. A small amount of ink should then be injected to verify placement. If placement is correct, the ink required for tattooing may be injected. The angle at which the needle is inserted into the mucosal wall is operator dependent, and may be influenced by peristalsis. Some endoscopists use a two-step approach by first injecting saline to create a bleb, and then using the same needle to inject the ink, in order to avoid transmural injection.

Advantageously, the hereindisclosed device allows a one-step procedure in which the bleb facilitating accurate submucosal injection is generated by the applying of the ring/loop-shaped tissue engagement member against the tissue to be tattooed, without requiring initial injection of saline, and allowing a non-operator dependent plain-limited injection.

The disclosure also provides an endoscopic guide tube configured to be inserted into a working channel of an endoscopic external cuff, which is folded upon insertion (as a single or multilayered fold) which may be expanded and/or unfolded at any point during the procedure. Endoscopic instrumentation may be used directly through the additional external working channel (single or multiple) formed by the cuff, or with the introduction of an additional guide tube made of or coated with a material having a low coefficient of friction, thus allowing essentially friction free or friction reduced passage of endoscopic accessory devices therethrough. Advantageously, the guide tube may be inserted into the working channel of the cuff only after the endoscope wearing the cuff has reached its target location, thereby ensuring an as easy and as comfortable as possible insertion of the endoscopic probe. This tube may encompass the entire circumference of the external working channel, or just the inner surface of its outer aspects, without covering its endoscope-adjacent aspects.

According to some embodiments, there is provided an endoscopic accessory device comprising an elongated shaft having a tissue engagement member at its distal end, the tissue engagement member configured to encircle gastrointestinal tissue when applied thereagainst.

According to some embodiments, the tissue engagement member may be rigid.

According to some embodiments, the tissue engagement member may be ring-shaped.

According to some embodiments, the tissue engagement member may have a first collapsed configuration configured for passage through an endoscope work channel and a second expanded configuration configured to encircle the gastrointestinal tissue.

According to some embodiments, the elongated shaft and the tissue engagement member are integrally formed.

According to some embodiments, the accessory device further includes a needle configured to deliver a fluid to the gastrointestinal tissue encircled by the tissue engagement member. According to some embodiments, the needle is configured to assume a first retracted position, and a second exposed position in which at least a sharp distal end of the needle extends into a space defined by the tissue engagement member.

According to some embodiments, the tissue engagement member may include one or more rubber bands configured to ligate an esophageal varix. According to some embodiments, the one or more rubber bands may encircle an outer or inner circumference of the tissue engagement member.

According to some embodiments, the accessory device may further include an expandable compartment configured to encompass the gastrointestinal tissue encircled by the tissue engagement member. According to some embodiments, the accessory device further includes an expansion member configured to induce expansion of the expandable compartment. According to some embodiments, the expandable compartment includes an inflatable wall. According to some embodiments, the accessory device further includes a suction member configured to induce a vacuum within the expandable compartment, thereby causing suction of the gastrointestinal tissue encircled by the tissue engagement member.

According to some embodiments, there is provided an endoscope accessory kit including: an endoscopic cuff configured to circumferentially encage at least part of an endoscope probe, the cuff including at least one working channel configured to allow passage of an endoscopic accessory device, wherein the cuff has a first collapsed and/or folded configuration and a second expanded/unfolded configuration; wherein the expanded/unfolded configuration is configured to allow passage of one or more endoscopic accessory devices through the at least one working channel; and a guide tube configured to be inserted into the working channel of the cuff, wherein the guide tube is made of a low friction material configured to allow essentially friction free or friction reduced passage of the one or more endoscopic accessory devices therethrough.

According to some embodiments, the kit further includes any of the endoscopic accessory devices disclosed herein.

According to some embodiments, there is provided a method for performing endoscopic tattooing, the method including: inserting an endoscopic accessory device through a working channel of an endoscope or of an endoscopic cuff, wherein the accessory device has an elongated shaft with a tissue engagement member at its distal end, and a needle; positioning the tissue engagement member on a gastrointestinal target tissue, such that the tissue engagement member encircles the target tissue; pressuring the tissue engagement member against the target tissue, such that the target tissue protrudes at least partially into a center space defined by the tissue engagement member; extending the needle from a retracted to an exposed position in which at least a sharp distal end of the needle extends into the space defined by the tissue engagement member, thereby piercing the protruding target tissue; and injecting an ink through the needle into the protruding target tissue.

According to some embodiments, the inserting of the endoscopic accessory device through the working channel of the endoscopic cuff comprises inserting the endoscopic accessory device through a guide member positioned within the cuff.

According to some embodiments, the method further includes inserting the guide member into the working channel of the cuff, thereby causing its expansion and/or unfolding.

According to some embodiments, there is provided a method for performing Endoscopic Variceal Ligation (EVL), the method including: inserting an endoscopic accessory device through a working channel of an endoscopic cuff, wherein the accessory device has an elongated shaft comprising a tissue engagement member at its distal end, and at least one elastic band configured for ligating an esophageal varix; positioning the tissue engagement member, such that the tissue engagement member encircles the esophageal varix; and releasing the at least one elastic band around the protruding esophageal varix.

According to some embodiments, the method may further include applying suction so as to cause the esophageal varix to protrude at least partially into a space defined by the tissue engagement member prior to the releasing of the at least one elastic band.

According to some embodiments, the endoscopic accessory device further includes an expandable compartment configured to encompass the gastrointestinal tissue encircled by the tissue engagement member. According to some embodiments, the method may further include expanding the compartment prior to the applying of the suction. According to some embodiments, expanding the compartment may include inflating a wall thereof using gas, fluid or mechanical struts.

According to some embodiments, inserting the endoscopic accessory device through the working channel of the endoscopic cuff may include inserting the endoscopic accessory device through a guide member positioned within the cuff.

According to some embodiments, the method further includes inserting the guide member into the working channel of the cuff, thereby causing its expansion and/or unfolding.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 shows a distal end of a conventional endoscope probe.

FIG. 2A and FIG. 2B schematically show a perspective and a front view, respectively, of an endoscope cuff with a collapsed working channel, according to some embodiments;

FIG. 2C and FIG. 2D schematically show a perspective and a front view, respectively, of an endoscope cuff with an expanded working channel, according to some embodiments;

FIG. 2E and FIG. 2F schematically show a perspective and a front view, respectively, of an endoscope cuff with a guide tube inserted through its working channel, according to some embodiments;

FIG. 2G and FIG. 2H schematically show a perspective and a front view, respectively, of an endoscope cuff with a guide tube inserted through its working channel, according to some embodiments;

FIG. 3 schematically shows an endoscope cuff having an endoscopic accessory device inserted through a guide tube inserted through the cuff working channel, according to some embodiments;

FIG. 4A and FIG. 4B schematically show an endoscope accessory device for endoscopic tattooing, including an elongated shaft, a tissue engagement member and a needle in a retracted and an exposed position respectively, according to some embodiments;

FIG. 5A to FIG. 5D schematically illustrate a method for endoscopic tattooing, according to some embodiments;

FIG. 6 schematically shows an endoscopic accessory device for esophageal varix ligation, according to some embodiments;

FIG. 7A to FIG. 7D schematically illustrate a method for esophageal varix ligation, according to some embodiments.

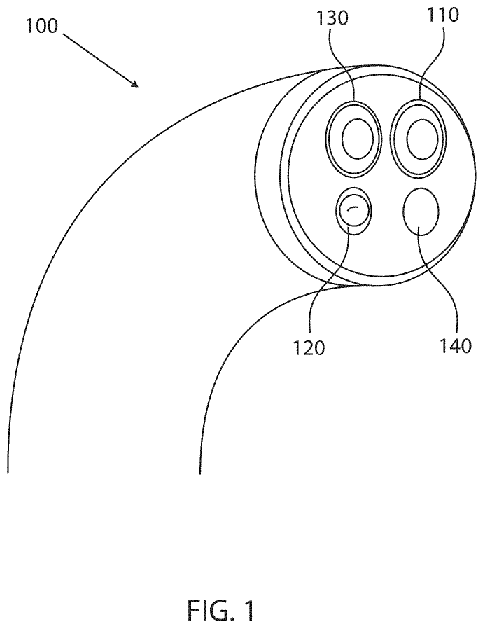

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. Additionally, it is to be explicitly understood that any combination of any one or more of the disclosed embodiments may be applicable and is within the scope of the disclosure.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

The present disclosure generally relates to the field of endoscopy, more particularly to endoscopic accessory devices.

According to some embodiments, there is provided an endoscopic accessory device having an elongated shaft with a tissue engagement member at its distal end, the tissue engagement member configured to encircle gastrointestinal tissue when applied there against. According to some embodiments, the elongated shaft and the tissue engagement member may be formed as an integral unit. According to some embodiments, the elongated shaft and the tissue engagement member may be produced in a single mold process. According to some embodiments, the tissue engagement member may be molded onto the distal end of the elongated shaft.

As used herein, the term “endoscopic accessory device” may refer to a device configured to be delivered to a target area through a working channel of an endoscopic probe or through a cuff encaging an endoscopic probe, as further elaborated herein. According to some embodiments, the accessory device may be configured to be passed through the working channel in its final configuration. Advantageously, the endoscopic accessory device may be retracted through the working channel of the endoscope or of the endoscope cuff, during the procedure without retracting the probe itself. Alternatively, the accessory device may be configured to assume its final configuration after exiting the working channel. According to some embodiments, the accessory device or parts thereof may be made of a memory shape material, such as, but not limited to, Nitinol™.

As used herein, the term “elongated shaft” may refer to part of the endoscopic accessory device allowing its maneuvering by a user, either directly or indirectly. According to some embodiments, the elongated shaft may be hollow along at least part of its length.

As used herein, the term “tissue engagement member” may refer to part of the endoscopic accessory device which is configured to encircle and thus confine, immobilize, bleb, or otherwise restrain a target tissue to allow further manipulation.

According to some embodiments, the tissue engagement member may be essentially ring-shaped. As used herein, the terms “ring-shaped” and “essentially ring-shaped” with regards to the tissue engagement member, may be used interchangeably and refer to any shape allowing to circumferentially encage the target tissue such as, but not limited to, a circular shape, an oval-shape, a loop shape or the like. According some embodiments, the tissue engagement member may be non-circular, i.e. having a rectangular or triangular shape or any other shape allowing to circumferentially enclose a target tissue.

According some embodiments, the tissue engagement member may be rigid. As used herein, the term “rigid” may refer to material applying counterforce when applied or pressed against gastrointestinal tissue. According some embodiments, the tissue engagement member may be partially flexible or adjustable, thus allowing the operator to modify the surface area encompassed by the member.

According to some embodiments, the tissue engagement member may be made of a material causing colon tissue to form a bleb protruding at least partially through the hollow center defined by the tissue engagement member (such as the center of the ring), when pushed/pressed there against.

According some embodiments, the tissue engagement member has a first collapsed configuration, coextensive with the elongated shaft, configured for passage through an endoscope work channel, and a second expanded (e.g. ring shaped) configuration configured to encircle the gastrointestinal tissue.

According to some embodiments, the inner diameter of tissue engagement member (at least in its second expanded configuration) may be larger than the diameter of the endoscopic probe. This advantageously facilitates encircling target tissues larger than the diameter of the endoscopic probe. This may be particularly advantageous, for example, when performing ligation of esophageal varices larger than can be encompassed by ligators typically used for performing the procedure.

According to some embodiments, the endoscopic accessory device may be for use in endoscopic tattooing. According some embodiments, the accessory device includes a needle or other piercing element/assembly capable of delivering a fluid, such as, but not limited to, an ink, into a gastrointestinal target tissue encircled by the tissue engagement member. According to some embodiments, the needle may be hollow and may be configured to pierce and deliver the fluid. Alternatively, a piercing assembly including a piercing element and a separate fluid delivery element may also be utilized.

According to some embodiments, the needle (or other piercing member/assembly) may be positioned such that its longitudinal axis is parallel to the longitudinal axis of the elongated shaft. According to some embodiments, the needle may be at least partially positioned within the lumen of the elongated shaft. According to some embodiments, the needle may be positioned along an outer surface of the elongated shaft.

According to some embodiments, the needle may be configured to assume a first retracted position, and a second exposed position in which at least a sharp distal end of the needle extends into a center space defined by the tissue engagement member. According to some embodiments, the elongated shaft and/or the needle may include a mechanism configured to cause the needle to move laterally, along its longitudinal axis, such that at least its sharp distal edge becomes positioned within the center confined by the circumference of the tissue engagement member, thus penetrating the tissue encircled by the tissue engagement member. According to some embodiments, the mechanism (same or different) may be configured to retract the needle so as to no longer extend into the center confined by the tissue engagement member upon completion of the injection of the fluid and/or upon completion of the procedure.

According to some embodiments, the endoscopic accessory device may be for use in esophageal varix ligation. According to some embodiments, the tissue engagement member may include one or more elastic bands, e.g. rubber bands, configured to ligate an esophageal varix. As used herein, the term “at least one”, with regards to elastic bands, may refer to 1, 2, 3, 3, 5, 6 or more elastic bands. Each possibility is a separate embodiment.

According to some embodiments, the tissue engagement member may include an indent on its inner or outer circumference, the indent configured to contain and preferably restrain the elastic band in an expanded configuration, wherein when released the elastic band shrinks to a size configured to tie the “neck” of the esophageal varix confined by the tissue engagement member.

Advantageously, since the endoscopic accessory device may be retracted through the working channel of the endoscope or the endoscope cuff during the procedure without retracting the probe itself, should additional elastic bands be required, the accessory device can be retracted, and a new one placed and operated, without a need for extraction of the entire scope, or it's re-introduction. According to some embodiments the endoscopic accessory device further includes an expandable compartment configured to encompass the esophageal tissue comprising the esophageal varix. As used herein, the term expandable compartment may refer to any compartment which may be expanded, and which upon expansion allows suction to be applied within the compartment without causing collapse thereof.

According to some embodiments, the expandable compartment includes an inflatable wall, which when inflated expands the compartment. According to some embodiments, the endoscopic accessory device further includes an inflation tube configured to trigger opening of the expandable compartment, for example, by inflating the wall of the expandable compartment, using gas, fluid, or mechanical struts. According to some embodiments, the inflation tube may be parallel to the elongated shaft. According to some embodiments, the inflation tube may be positioned within the lumen of the elongated shaft. According to some embodiments, the inflation tube may be adjacent to but external to the elongated shaft. According to some embodiments, the distal end of the inflation tube may be distal to the distal end of the elongated shaft. According to some embodiments, the distal end of the inflation tube may extend into the expandable compartment.

According to some embodiments, the compartment includes an expandable skeleton/cage, which, when deployed, expands the compartment.

According to some embodiments, the compartment is configured to enclose the encircled esophageal tissue in such manner that, if suction is applied, a vacuum is created within the compartment causing the esophageal tissue to bleb through the tissue engagement member, as further illustrated herein. According to some embodiments, the elongated shaft may also serve as a suction tube. Alternatively, the accessory device may further include a suction tube. According to some embodiments, the suction tube may be parallel to the elongated shaft. According to some embodiments, the suction tube may be positioned within the lumen of the elongated shaft. According to some embodiments, the suction tube may be adjacent to but external to the elongated shaft. According to some embodiments, the distal end of the suction tube may be distal to the distal end of the elongated shaft. According to some embodiments, the distal end of the suction tube may extend into the expandable compartment.

According to some embodiments, there is provided an endoscope accessory kit including an endoscopic cuff configured to circumferentially encage at least part of an endoscope probe, the cuff including at least one working channel configured to allow passage of an endoscopic accessory device, wherein the cuff has a first collapsed or folded (single layered or multiple-layered fold) configuration and a second expanded configuration; wherein the expanded configuration is configured to allow passage of one or more endoscopic accessory devices through the at least one working channel; and a guide tube configured to be inserted into the working channel, wherein the guide tube is configured to allow essentially friction free or friction reduced passage of the one or more endoscopic accessory devices therethrough.

As used herein, the terms “cuff”, “sheath” and “overtube” may be used interchangeably and refer to a sheath-like layer of material configured to cover or be draped over an endoscopic probe. The endoscopic probe may be completely or partially enclosed by the external cuff.

As used herein, the terms “guide tube” and “guide member” may be interchangeably used and may refer to any elongated guide member configured to be positioned within the working channel, thereby allowing essentially friction free or friction reduced passage of accessory devices therethrough.

According to some embodiments, the cuff may be used with endoscopic probes of various sizes, such as, but not limited to, colonoscopes, gastroscopes, side-viewing scopes, endoscopic ultrasound scopes, adult and pediatric scopes, rigid and flexible scopes and single and multi-channel scopes. According to some embodiments, the cuff may fit and/or be suitable for use with non-GI scoping instrumentation.

According to some embodiments, the material forming the cuff may be flexible.

According to some embodiments, the working channel may extend along the entire length of the endoscope probe. According to some embodiments, the working channel(s) may extend along part of the length of the endoscope probe. According to some embodiments, the working channel(s) may be essentially parallel to the working channel of the endoscope probe. According to some embodiments, the working channel(s) may be angled relative to the endoscope probe, at least along part of its length. As a non-limiting example, the working channel(s) may exit in an upward angle.

According to some embodiments, the cuff may include a single (no more than one) working channel. According to some embodiments, the working channel may extend along the entire circumference of the cuff. According to some embodiments, the working channel may extend along part of the cuff's circumference, thus forming a “banana shaped” working channel, as essentially illustrated herein.

According to some embodiments, the cuff may include more than one working channel, such as 2, 3, 4, 5 or more working channels. Each possibility is a separate embodiment.

According to some embodiments, the cuff may include a plurality of working channels. According to some embodiments, the cuff may include at least two working channels. According to some embodiments, the at least two working channels may have a same size and/or shape. According to some embodiments, the at least two working channels may have a different size and/or shape. According to some embodiments, each of the working channels may be circumferentially spaced around the endoscopic probe, thereby permitting a combination of endoscopic accessory devices to be used in co-operation with each other to perform a medical procedure.

According to some embodiments, the working channel may terminate in a distal end opening. According to some embodiments, working channels may include one or more apertures/openings positioned proximally to the distal end, such as, but not limited to, 1-10 cm from the distal end of the cuff, which one or more apertures/openings are configured to allow accessory devices, medical instruments, and/or imaging probes to exit/be retracted therethrough.

According to some embodiments, the outer surface of the cuff may be configured to allow essentially smooth access of the cuff to tight tissue locations. That is, the outer surface of the cuff may, according to some embodiments, be made of a lubricious material configured to allow smooth passage, e.g. a material allowing the endoscopic probe, covered by the cuff, to essentially glide in to a desired tissue location, i.e. the rectum of a subject. Additionally or alternatively, the outer surface may be covered by an additional lubricious layer configured to allow smooth access of the endoscopic probe, covered by the cuff. According to some embodiments, the additional layer may be an integral part of the cuff, be provided with the cuff or be applied on the cuff prior to use (e.g. after having been applied on the probe, but prior to insertion).

According to some embodiments, the inner surface of the cuff is configured to circumferentially encase/engage/enclose/sheath an endoscopic probe. According to some embodiments, the inner surface of the cuff may be made of a material configured to allow the cuff to be easily applied on the probe.

According to some embodiments, the inner surface of the cuff may be made of a material configured to ensure friction compression fit of the cuff to the probe, i.e. to prevent the cuff from moving relative to the probe once applied thereon. Additionally or alternatively, the inner surface may be covered by an additional layer configured to ensure friction compression fit of the cuff to the probe. According to some embodiments, the additional layer may be an integral part of the cuff, be provided with the cuff or be applied on the cuff prior to use. Each possibility is a separate embodiment.

According to some embodiments, the cuff may be sized and shaped to prevent the cuff from moving relative to the endoscope probe once applied thereon.

According to some embodiments, the cuff may include an attachment assembly configured to engage the distal tip of the endoscopic probe, thereby preventing the probe from passing through the distal end of the cuff and/or preventing the cuff from retracting/rolling back during forward movement/insertion of the probe into the body cavity/organ lumen. According to some embodiments, the attachment assembly may be in a shape of the cuff. For example, the cuff may be tapered in circumference such that the circumference at the distal end of the cuff is somewhat smaller than the circumference of the distal end of the probe. According to some embodiments, the attachment assembly may be an element such as a clip, a hook, a button or any other suitable element affixed to or formed with the distal end of the endoscopic probe and configured to prevent the probe from passing through the distal end of the cuff and/or preventing the cuff from retracting/rolling back during forward movement/insertion of the probe into the body cavity/organ lumen.

According to some embodiments, the cuff has a first collapsed configuration. According to some embodiments, in its collapsed configuration the outer diameter of the cuff encaging/engaging the endoscopic probe is below 15 mm, below 14 mm, below 13 mm, below 12.5 mm, below 12 mm, below 11.5 mm, below 11 mm, below 10.5 mm or below 10 mm. Each possibility is a separate embodiment. According to some embodiments, the outer diameter of the endoscope probe with the cuff in its collapsed configuration is increased by less than 2%, less than 3%, less than 5%, less than 10%, less than 15%, less than 20%, less than 25% or less than 30% as compared to the outer diameter of the endoscopic probe and/or shaft itself. Each possibility is a separate embodiment. According to some embodiments, the circumference of the cuff may be adjusted to fit endoscope probes of various sizes and/or shapes. For example, some cuffs may be provided with a circumference configured to fit endoscopes used in adults, while others are made to fit pediatric use.

According to some embodiments, the cuff has a first folded configuration. This can be a single-layered fold or a multiple-layered fold; both can partially or completely cover the endoscope circumference.

According to some embodiments, in the expanded configuration, passage of one or more endoscopic accessory devices through the working channel is possible.

According to some embodiments, the cuff may be self-expandable. According to some embodiments, the cuff may be configured to expand (automatically) once the probe reaches its target location, for example, due to the temperature and/or humidity prevailing at the target location. According to some embodiments, the cuff may be made of or include a shape memory material, such as a memory shape alloy or a memory shape polymer. Each possibility is a separate embodiment. According to some embodiments, the memory shape material may be configured to cause expansion of the cuff once reaching the target location.

Additionally or alternatively, the cuff may be expanded “on-the fly” i.e. by insertion/when passing the guide tube, through the working channel.

Additionally or alternatively, the cuff may be expanded by activating an expansion mechanism.

According to some embodiments, expansion of the cuff may increase the outer diameter of the cuff while leaving the inner diameter of the cuff essentially unaffected, thereby increasing the diameter of the working channel. As a non-limiting example, passage of the guide tube through the working channel may cause the roof of the working channel to be lifted/stretched, thereby increasing the diameter thereof.

According to some embodiments, the cuff may resume its collapsed configuration when the guide tube is pulled out of the longitudinal bores. According to some embodiments, the cuff may resume its collapsed configuration as a result of backward movement of the probe, i.e. as a result of its retrieval. According to some embodiments, the cuff may resume its collapsed configuration as a result of the backward movement of the guide tube, i.e. as a result of its withdrawal. According to some embodiments, the cuff may resume its collapsed configuration due to deactivation of the expansion mechanism and/or due to activation of a collapse mechanism.

According to some embodiments, the guide member may be inserted into the working channel after the endoscope has accessed its target location or after the endoscope has entered a tight tissue location but prior to reaching its target location. According to some embodiments, once inserted into the working channel, the guide tube is essentially stably positioned within the working channel, i.e. backward/forward gliding of the guide member is essentially avoided. According to some embodiments, the working channel may close around the guide member, thus essentially immobilizing it therein.

According to some embodiments, the guide member may have a shape of a cylinder's sector or of a gutter, as essentially illustrated herein. According to some embodiments, the guide member may have a shape of a closed half-cylinder, as essentially illustrated herein.

According to some embodiments, at least the inner surface of the guide member may be made or covered with a material having a low friction coefficient, so as to allow essentially friction free/essentially unhindered passage of endoscopic accessory devices through the working channel of the cuff. As used herein, the term “low” with regards to the friction coefficient may refer to a friction coefficient below 1, below 0.8, below 0.7, below 0.6 or below 0.5. Each possibility is a separate embodiment.

According to some embodiments, insertion of the guide member may be configured to cause the cuff to assume its expanded configuration.

According to some embodiments, insertion of the guide member may be configured to cause the cuff to assume its unfolded configuration.

According to some embodiments, the kit may further include an endoscopic accessory device, such as, but not limited to, any of the endoscopic accessory devices disclosed herein.

According to some embodiments, there is provided a method for performing endoscopic tattooing in a subject in need thereof, the method including:
(a) inserting an endoscopic accessory device through a working channel of an endoscope or of an endoscopic cuff, wherein the accessory device includes an elongated shaft with an optionally essentially ring-shaped tissue engagement member at its distal end and a needle having a longitudinal axis parallel to the longitudinal axis of the elongated shaft;
(b) positioning the tissue engagement member on/against a gastrointestinal target tissue (e.g. a colorectal lesion), such that the tissue engagement member encircles the target tissue;
(c) pressuring the tissue engagement member against the target tissue such that the target tissue protrudes at least partially into a center space defined by the tissue engagement member. It is understood that the applying of the tissue engagement member against the target tissue may be concurrent with the step of positioning the tissue engagement member. Alternatively, the tissue engagement member may initially be positioned at a target location and, only once the location is confirmed as correct, pressure is applied on the tissue engagement member thereby causing the target tissue to protrude/bleb through the tissue engagement member;
(d) extending the needle from a retracted to an exposed position, such that at least the sharp distal end of the needle extends into the center space defined by the tissue engagement member, thereby penetrating into the target tissue confined/encircled by the tissue engagement member; and
(e) injecting an ink, dye, or other material through the needle into the target tissue. It is understood that injecting the ink may include initially injecting a small bolus of ink to verify correct injection. Alternatively, the entire amount of ink may be injected in a single bolus.

As used herein, the terms “patient” and “subject” may be interchangeably used and may refer to any subject undergoing an endoscopic procedure.

According to some embodiments, the endoscopic accessory device may be for use in endoscopic tattooing, described herein.

According some embodiments, the tissue engagement member may have a first collapsed configuration, coextensive with the elongated shaft, configured for passage through an endoscope work channel, and a second expanded (e.g. ring shaped) configuration configured to encircle the gastrointestinal tissue. According to some embodiments, the method may further include having the tissue engagement member assume its second expanded configuration (passively or actively) upon exiting the distal end of the working channel.

According to some embodiments, the method further includes withdrawing the endoscopic accessory device upon completion of the procedure.

According to some embodiments, there is provided a method for performing Endoscopic Variceal Ligation (EVL) in a subject in need thereof, the method comprising:
(a) inserting an endoscopic accessory device through a working channel of an endoscope or of an endoscopic cuff, wherein the accessory device has an elongated shaft with an optionally essentially ring-shaped tissue engagement member at its distal end, and at least one band configured for ligating an esophageal varix;
(b) positioning the endoscopic accessory device, such that the ring-shaped tissue engagement member encircles the esophageal varix; and
(c) releasing the at least one band around the protruding esophageal varix, such that the band ties off the esophageal varix. It is understood that the more than one band may be released simultaneously or sequentially.

It is understood that, should additional bands be needed, the method may further include withdrawing the endoscopic accessory device, inserting a new endoscopic accessory device through the working channel and releasing the additional band(s) around the tissue, thereby further tying off the esophageal varix or others without extracting and re-inserting the scope.

According to some embodiments, the method may further include applying suction so as to cause the esophageal varix to protrude at least partially into a center space defined by the tissue engagement member, prior to the releasing of the at least one band.

According to some embodiments, the endoscopic accessory device may be the endoscopic accessory device for esophageal varix ligation, described herein.

According to some embodiments, the endoscopic accessory device may include an expandable compartment configured to create a chamber to encompass the gastrointestinal tissue encircled by the tissue engagement member. According to some embodiments, the method may further include expanding the chamber prior to the applying of the suction. According to some embodiments, expanding the compartment may including inflating (using gas, fluid or mechanical struts) the wall of the compartment.

According some embodiments, the tissue engagement member may have a first collapsed configuration, coextensive with the elongated shaft, configured for passage through an endoscope work channel, and a second expanded (e.g. ring shaped) configuration configured to encircle the gastrointestinal tissue. According to some embodiments, the method may further include having the tissue engagement member assume its second expanded configuration (passively or actively) upon exiting the distal end of the working channel.

According to some embodiments, the method further includes withdrawing the endoscopic accessory device upon completion of the procedure.

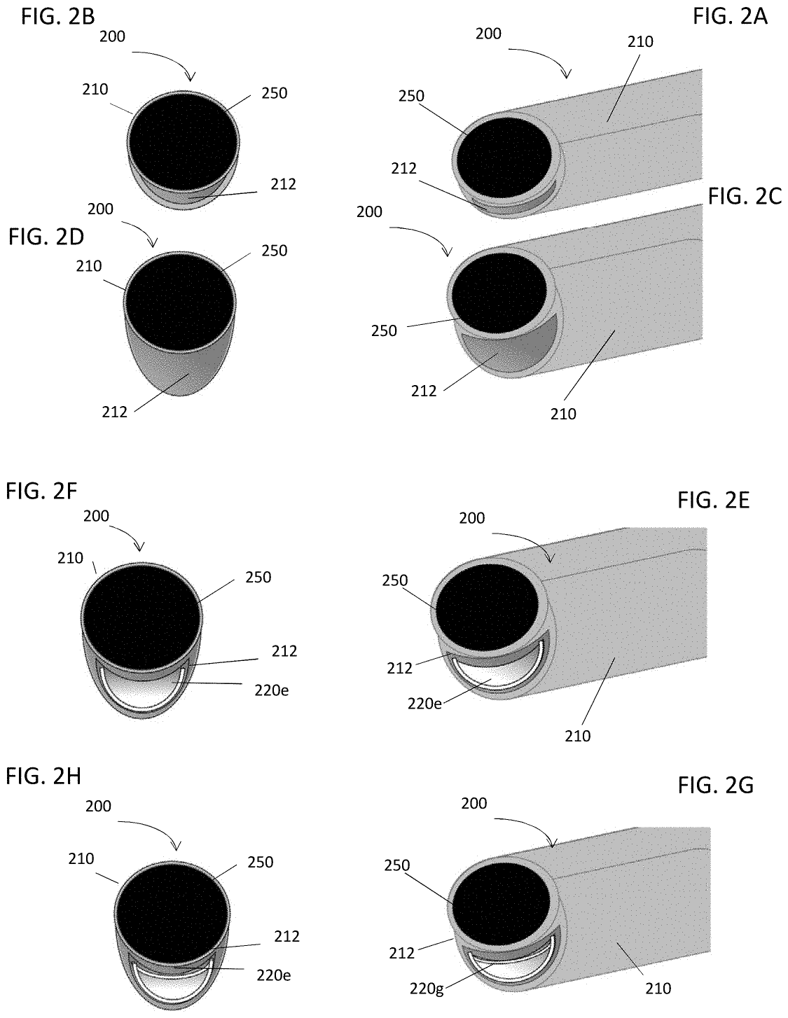

Reference is now made to FIG. 2A and FIG. 2B which schematically show a perspective and a front view, respectively, of an endoscope assembly 200 including an endoscope cuff 210, draped around an endoscope probe 250, with a collapsed working channel 212, according to some embodiments. In the collapsed configuration, working channel 212 is collapsed, such that the diameter of endoscope probe 250 covered by cuff 210 is only slightly increased vis-à-vis the diameter of endoscope probe 250 alone, thereby ensuring relatively easy entry of endoscope assembly 200 into a body cavity/organ lumen (not shown) of a patient. According to some embodiments, endoscope cuff 210 may include a lubricious material on its outer surface, the lubricious material configured to provide essentially smooth insertion of endoscope assembly 200 into the patient's body cavity/organ lumen. According to some embodiments, endoscopic cuff 210 may be tapered at its distal end, such that the circumference of endoscope cuff 210 is smaller than the circumference of the distal end of endoscope probe 250. Additionally or alternatively, endoscope cuff 210 may include an attachment mechanism configured to grasp the distal end of endoscope probe 250 thereby preventing retraction/folding back of endoscope cuff 210 relative to endoscope probe 250, during insertion. According to some embodiments, the cuff (210) is folded in the resting position, in a single layer or multiple layer fashion.

Reference is now made to FIG. 2C and FIG. 2D which schematically show a perspective and a front view, respectively, of an endoscope assembly 200 including an endoscope cuff 210, draped around an endoscope probe 250, with an expanded working channel 212, according to some embodiments. In the expanded configuration, working channel 212 allows passage of endoscopic accessory devices, as essentially described herein. Advantageously, working channel 212 may be expanded only when endoscopic assembly 200 reaches its target location so as not to interfere with the introduction of endoscope assembly 200 into the patient's gastrointestinal lumen (or non-GI lumen and other body cavities). According to some embodiments, expansion of working channel 212 may be obtained as a result of insertion and passage of a guide member, as further elaborated herein, and may collapse with the withdrawal of the guide member. Additionally or alternatively, the expansion of working channel 212 may be by activating an expansion mechanism (e.g. inflating a balloon within the bore), as essentially described herein.

Reference is now made to FIG. 2E and FIG. 2F which schematically show a perspective and a front view, respectively, of an endoscope assembly 200 including an endoscope cuff 210, draped around an endoscope probe 250, wherein endoscope cuff 210 has a guide member 220e inserted through its working channel 212. Guide member 220e is configured to allow essentially friction free or friction reduced passage of the one or more endoscopic accessory devices (not shown) therethrough, and is here shown as having a shape of a cylinder's sector, thus providing a gutter-like slide for friction-free insertion of an accessory device. Guide member 220e may be inserted into working channel 212 after endoscope probe 250 has been inserted. Once inserted into working channel 212, guide member 220e is stabilized or essentially immobilized within working channel 212, i.e. backward/forward gliding of guide member 220e is essentially avoided (while still allowing withdrawal and reinsertion of guide member 220e if needed). At least the inner surface 222 of guide member 220e may be made or covered with a material having a low friction coefficient, so as to allow essentially friction free or friction reduced/essentially unhindered passage of endoscopic accessory devices (not shown) through working channel 212 of cuff 210. According to some embodiments, insertion of guide member 220e may be configured to cause cuff 210 to assume its expanded configuration. Guide member 220e may also allow for suction bowel contents, including particles and resected tissue too large to be suctioned through the regular suction channel within the scope.

Reference is now made to FIG. 2G and FIG. 211 which schematically show a perspective and a front view, respectively, of an endoscope assembly 200 including an endoscope cuff 210, draped around an endoscope probe 250, wherein endoscope cuff 210 has a guide member 220g inserted through its working channel 212. Guide member 220g is configured to allow essentially friction free or friction reduced passage of the one or more endoscopic accessory devices (not shown) therethrough and is here shown as having a shape of a closed half-cylinder, thus providing a channel for friction-free insertion of an accessory device. Guide member 220g may be inserted into working channel 212 after endoscope probe 250 has been inserted. Once inserted into working channel 212, guide member 220g is stabilized or essentially immobilized within working channel 212, i.e. backward/forward gliding of guide member 220g is essentially avoided (while still allowing withdrawal and reinsertion of guide member 220e if needed). At least the inner surface 222 of guide member 220g may be made or covered with a material having a low friction coefficient, so as to allow essentially friction free or friction reduced or essentially unhindered passage of endoscopic accessory devices (not shown) through working channel 212 of cuff 210. According to some embodiments, insertion of guide member 220g may be configured to cause cuff 210 to assume its expanded configuration.

Guide member 220g may also allow for suction bowel contents, including particles and resected tissue too large to be suctioned through the regular suction channel within the scope.

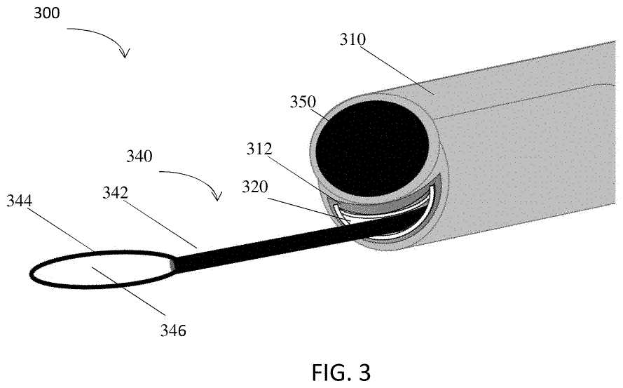

Reference is now made to FIG. 3, which schematically shows an endoscope assembly 300 including an endoscope cuff 310, draped around an endoscope probe 350, endoscope cuff 300 having an endoscopic accessory device 340 inserted through a guide member 320 positioned within working channel 312 of cuff 310, according to some embodiments. Endoscopic accessory device 340 includes an elongated shaft 342 having a tissue engagement member 344 at its distal end. Tissue engagement member 344 is configured to encircle gastrointestinal tissue (not shown) when applied there against. According to some embodiments, accessory device 340 may be configured to be passed through working channel 312 via guide member 320 in its final configuration. Alternatively, tissue engagement member 344 may initially assume a collapsed/folded configuration (not shown) allowing unhindered/easy access/passage through working channel 312 via guide member 320, whereafter, upon exiting working channel 312, tissue engagement member 344 assumes its essentially ring/loop-shaped configuration, illustrated in FIG. 3. Advantageously, endoscopic accessory device 340 may be retracted through working channel 312, during the procedure without retracting endoscope probe 350 itself. According to some embodiments, tissue engagement member 344 may be made of a material causing colon tissue to form a bleb protruding at least partially through the hollow center 346 defined by tissue engagement member 344, when pushed/pressed against the tissue. According to some embodiments, the diameter of tissue engagement member 344 (at least in its second expanded configuration) may be larger than the diameter of endoscopic probe.

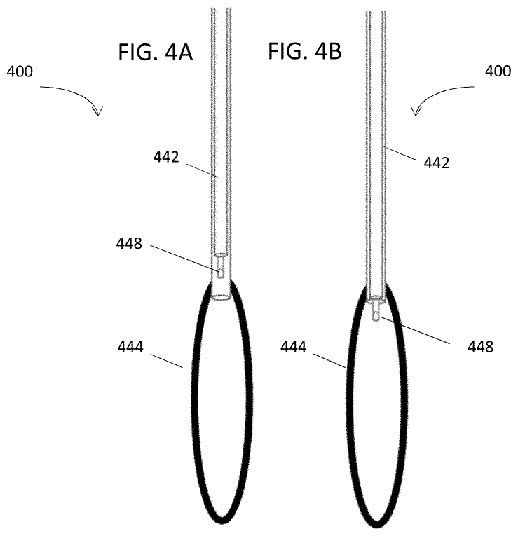

Reference is now made to FIG. 4A and FIG. 4B, which schematically show an endoscope accessory device for endoscopic tattooing 400, including an elongated shaft 442, a tissue engagement member 444 and a needle 448 in a retracted (FIG. 4A) and an exposed (FIG. 4B) position, according to some embodiments. Optionally, elongated shaft 442 may be hollow at least along a portion of its length and may allow needle 448 to be positioned within its lumen in essentially in its entirety, in the retracted position of needle 448 or partially, in the exposed position of needle 448. Needle 448 may be configured to change into its exposed position by forward movement of needle 448 along the longitudinal axis defined by elongated shaft 442. According to some embodiments, the needle and engagement member are angled relative to the longitudinal axis of the elongated shaft. Needle 448 may optionally be hollow and may be configured to deliver a fluid such as ink to the tissue into which it has penetrated.

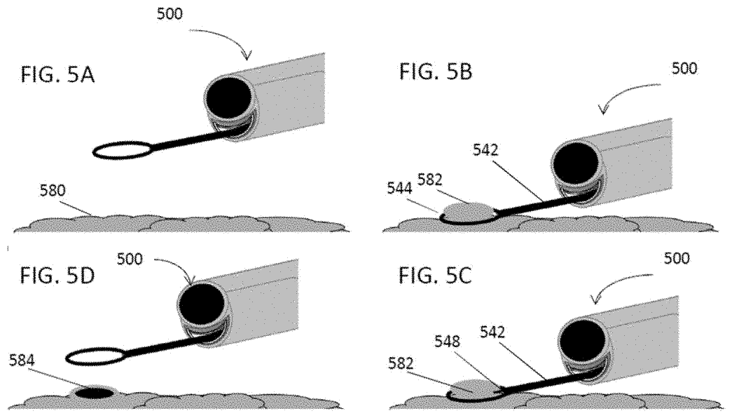

Reference has is now made to FIG. 5A to FIG. 5D, which schematically illustrate a method 500 for endoscopic tattooing, according to some embodiments. As seen in FIG. 5A, the method includes inserting an endoscopic accessory device 500, which may be essentially identical to endoscopic accessory assembly 340 and 400 described hereinabove, through a working channel (such as working channel 112 optionally via a guide member such as guide member 320) of an endoscopic cuff (which may be essentially similar to endoscope cuff 210 and 310). In a following step, illustrated in FIG. 5B, the method includes positioning the tissue engagement member 544 of endoscopic accessory device 500 on/against a gastrointestinal target tissue 580 (e.g. a colorectal lesion), such that tissue engagement member 544 encircles target tissue 580 and causes it to bleb 582 through the hollow center of tissue engagement member 544. In a following step, illustrated in FIG. 5C, a needle 548 may be moved laterally, along the longitudinal axis defined by elongated shaft 542, thereby causing needle 548 to penetrate into blebbed target tissue 582, encircled by tissue engagement member 544 and ink is injected. Upon, injection of the ink, tissue engagement member 544 may release the tattooed target tissue, as illustrated in FIG. 5D and endoscopic accessory device 500 can be withdrawn through working channel 112, leaving a visible mucosal marking 584.

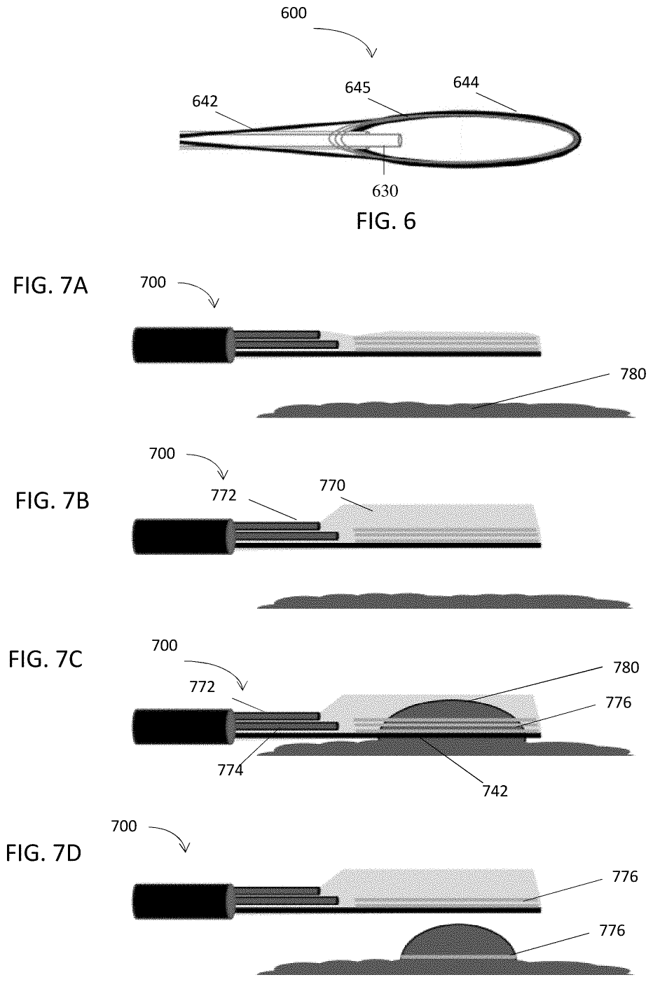

Reference is now made to FIG. 6 which schematically show an endoscope accessory device 600 for esophageal varix ligation, including an elongated shaft 642, a tissue engagement member 644 and one or more elastic bands 645. Accessory device 600 also includes a suction tube 630 configured to suck tissue encircled by tissue engagement member 644, thereby causing the encircled tissue to be sucked there through. According to some embodiments, accessory device 600 may further include an expandable compartment (not shown) configured to encompass the tissue suctioned through tissue engagement member 644. According to some embodiments, an expandable compartment may refer to any compartment which may be expanded, and which upon expansion allows suction to be applied within the compartment without causing its collapse. For example, the expandable compartment may have an inflatable wall, which when inflated expands the compartment using gas, fluid or mechanical struts (each possibility is a different embodiment). According to some embodiments, endoscopic accessory device 600 may further include an inflation tube (not shown) configured to trigger expansion of the expandable compartment, for example, by inflating its wall. It is understood that the assembly, including the expandable compartment, the inflation tube and suction tube 630, generates an isolated chamber allowing suctioning of mucosa, such as esophageal tissue, through the “loop-hole” of tissue engagement member 644, which in turn enables it to tie off the base of the suctioned tissue by releasing elastic bands therearound.

Reference is now made to FIG. 7A to FIG. 7D, which schematically illustrate a method for esophageal varix ligation, according to some embodiments. As seen in FIG. 7A, the method includes inserting an endoscopic accessory device 700 through a working channel of an endoscope or of an endoscopic cuff, which may be essentially identical to endoscopic accessory device 700 described hereinabove, through a working channel (such as working channel 112 optionally via a guide member such as guide member 320) of an endoscopic cuff (which may be essentially similar to endoscope cuff 210 and 310). In a following step, illustrated in FIG. 7B, the method includes expanding an expandable compartment 770, by inflating the wall of the compartment using inflation tube 772. After expansion of compartment 770, suction may be applied within compartment 770 using suction tube 774. As a result, target tissue 780 becomes sucked through the loop of tissue engagement member 742, as illustrated in FIG. 7C. Once a bleb is formed, one or more elastic bands 776 may be released around target tissue 780 so as to tie it off and enable ligation of its esophageal varix, as seen in FIG. 7C and FIG. 7D. It is understood that the number of elastic bands released may depend on the anatomy of esophageal varix, clinical outcome, patient and operator related factors etc.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-24. (canceled)

25. An endoscopic accessory device comprising an elongated shaft comprising a tissue engagement member at its distal end, said tissue engagement member configured to encircle gastrointestinal tissue when applied thereagainst.

26. The accessory device of claim 25, wherein said tissue engagement member has a first collapsed configuration configured for passage through an endoscope work channel, and a second expanded configuration configured to encircle the gastrointestinal tissue.

27. The accessory device of claim 25, wherein said elongated shaft and said tissue engagement member are integrally formed.

28. The accessory device of claim 25, further comprising a needle configured to deliver a fluid to the gastrointestinal tissue encircled by said tissue engagement member, preferably.

29. The accessory device of claim 28, wherein said needle is configured to assume a first retracted position, and a second exposed position in which at least a sharp distal end of said needle extends into a space defined by said tissue engagement member.

30. The accessory device of claim 25, wherein said tissue engagement member comprises one or more elastic bands configured to ligate an esophageal varix.

31. The accessory device of claim 30, wherein said one or more elastic bands encircle an outer or inner circumference of said tissue engagement member.

32. The accessory device of claim 30, further comprising an expandable compartment configured to encompass the gastrointestinal tissue encircled by said tissue engagement member.

33. The accessory device of claim 32, further comprising a suction member configured to induce a vacuum within said expandable compartment, thereby causing suction of the gastrointestinal tissue encircled by said tissue engagement member.

34. A method for performing endoscopic tattooing, the method comprising:
inserting an endoscopic accessory device through a working channel of an endoscope or of an endoscopic cuff, wherein the accessory device comprises an elongated shaft comprising a tissue engagement member at its distal end and a needle;
positioning the tissue engagement member on a gastrointestinal target tissue, such that the tissue engagement member encircles the target tissue;
pressuring the tissue engagement member against the target tissue such that the target tissue protrudes at least partially into a space defined by said tissue engagement member;
extending the needle from a retracted to an exposed position in which at least a sharp distal end of said needle extends into the space defined by the tissue engagement member thereby piercing the protruding target tissue; and
injecting an ink through said needle into the protruding target tissue.

35. The method of claim 34, wherein the inserting of the endoscopic accessory device through the working channel of the endoscopic cuff comprises inserting the endoscopic accessory device through a guide member positioned within the cuff.

36. The method of claim 35, further comprising inserting the guide member into the working channel of the cuff, thereby causing its expansion and/or unfolding.

37. A method for performing Endoscopic Variceal Ligation (EVL), the method comprising:
inserting an endoscopic accessory device through a working channel of an endoscopic cuff, wherein the accessory device comprises an elongated shaft comprising a tissue engagement member at its distal end, and at least one elastic band configured for ligating an esophageal varix;
positioning the tissue engagement member, such that the tissue engagement member encircles the esophageal varix; and
releasing the at least one elastic band around the protruding esophageal varix.

38. The method of claim 37, further comprising applying suction to cause the esophageal varix to protrude at least partially into a space defined by the tissue engagement member prior to the releasing of the at least one elastic band.

39. The method of claim 38, wherein the endoscopic accessory device further comprises an expandable compartment configured to encompass the gastrointestinal tissue encircled by the tissue engagement member and wherein the method comprises expanding the compartment prior to the applying of the suction.

40. The method of claim 39, wherein expanding the compartment comprises inflating a wall thereof using gas, fluid or mechanical struts.

41. The method of claim 37, wherein the inserting the endoscopic accessory device through the working channel of the endoscopic cuff comprises inserting the endoscopic accessory device through a guide member positioned within the cuff.

42. The method of claim 41, further comprising inserting the guide member into the working channel of the cuff, thereby causing its expansion and/or unfolding.
